(19) United States
(12) Patent Application Publication
(10) Pub. No.: US 2017/0246351 A1
(43) Pub. Date: Aug. 31, 2017

(54) Biocompatible Textile Sleeves to Support and Guide Muscle Regeneration and Methods of Use Thereof

(71) Applicants: Rutgers, The State University of New Jersey, New Brunswick, NJ (US); The General Hospital Corporation DBA Massachusetts General Hospital, Boston, MA (US)

(72) Inventors: N. Sanjeeva MURTHY, Princeton, NJ (US); Joachim B. KOHN, Piscataway, NJ (US); Cathryn SUNDBACK, Harvard, MA (US); Craig M. NEVILLE, Melrose, MA (US)

(21) Appl. No.: 15/508,349

(22) PCT Filed: Sep. 2, 2015
(86) PCT No.: PCT/US15/48105

§ 371 Date: Mar. 2, 2017

Related U.S. Application Data
(60) Provisional application No. 62/044,499, filed on Sep. 2, 2014.

(51) Int. Cl. A61L 27/58

(52) U.S. Cl. CPC A61L 27/58; A61L 2400/16; A61L 2430/30; A61F 2002/0894

(57) ABSTRACT

A biocompatible sleeve designed to encase an assembly of small 3D muscles that have been cultured in vitro. The sleeve is formed from polymer fibers in such a way that pushing the two ends of the sleeve towards each other increases the diameter of the sleeve so as to facilitate insertion of the engineered muscles. Subsequent pulling at the ends of the sleeves decreases the diameter of the sleeve to facilitate a secure fit around the engineered muscle during implantation of the sleeve into a patient. The composition of the polymer fibers can be tuned to achieve the desired mechanical properties and rate of degradability.

### CROSS-REFERENCE TO RELATED APPLICATIONS

This application claims the benefit of priority of U.S. Provisional Application No. 62/044,499, filed on Sep. 2, 2014, the entire disclosure of which is incorporated hereby by reference.

### STATEMENT REGARDING FEDERALLY SPONSORED RESEARCH

The invention described herein was supported in whole or in part by Grant No. 431934 awarded by the Congressional Directed Medical Research Programs of the U.S. Department of Defense. The U.S. Government has certain rights in the invention.

### FIELD OF THE INVENTION

The present invention relates to implantable muscles for the repair of injured or diseased muscles, and more particularly to textile sleeves for use with engineered muscle constructs which support physiological connection to a patient's neurovascular network.

### BACKGROUND OF THE INVENTION

Reconstruction of skeletal muscle tissue lost by traumatic injury, tumor ablation, or functional damage due to myopathies is hindered by the lack of available functional muscle tissue substitutes. Muscle transplantation or transposition techniques provide a limited degree of functional restoration.

One approach to addressing muscle tissue reconstruction is to engineer new tissue. Scaffolds for engineering such muscle tissues have been produced by several methods and tested in the laboratory. Generally, these scaffolds are seeded with muscle progenitor cells prior to implantation. Various methods for producing muscle tissue are known in the art. There is no existing technology commercially available, however, to reconstruct skeletal muscle tissue or restore muscle function through the use of fully formed engineered muscles prior to implantation. Most recently, stem cells have been injected into sites of muscle damage to successfully regenerate small areas of damage. Decellularized extracellular matrices have been used to fill areas of large volumetric muscle loss with some limited return of function.

Accordingly, there is a need in the art for an implantation sleeve and methods of use of same that provide both construct strength for implantation and mechanical support to fully formed muscle tissue until the implanted tissue has sufficient mechanical integrity, among other desirable features, as described herein.

### SUMMARY OF THE INVENTION

The present invention is related to engineering muscle which promote physiological connection to the patient's (i.e., human or animal) neurovascular network using a flexible sleeve to encase a plurality of muscle constructs for implantation, and using polymer fibers with tunable degradation characteristics. The sleeve provides the strength for the muscle constructs to be bundled and implanted and is permeable to allow patient vascular ingrowth and to support the transferred patient nerve which will innervate the implanted muscle. These features enable the implant to be vascularized and innervated. By the time the muscle is fully functional, the sleeve degrades and is reabsorbed by the body.

In at least one embodiment, the present invention provides a biocompatible sleeve for use with an implantable tissue, which is fabricated from a resorbable biocompatible polymer. This resorbable biocompatible polymer is configured to have a stiffness similar to that of muscle fibers, to have shape memory and a specific degradation profile.

In certain embodiments, the sleeve may also include a textured polymer fiber.

In certain embodiments, the sleeve may assume the form of a cylindrical shape.

In certain embodiments, the implantable tissue may be muscle.

In certain embodiments, the sleeve may further include a polymer or collagen gel.

In certain embodiments, the sleeve may be coated with an electrospun mat.

In certain embodiments, the sleeve may also include a second polymer that has a different degradation profile than the first polymer.

In certain embodiments, the sleeve may include small molecules, bioreactive compounds, and/or proteins.

In at least one embodiment, the present invention provides a method of using a biocompatible sleeve to support and guide muscle regeneration.

Various advantages of this invention will become apparent to those skilled in the art from the following detailed description of the invention, when read in light of the accompanying drawings.

### BRIEF DESCRIPTION OF THE DRAWINGS

FIG. 1 is a schematic of an exemplary implant sleeve in accordance with an exemplary embodiment of the invention.

FIG. 2 is a schematic of an exemplary implant sleeve in accordance with another exemplary embodiment of the invention in a non-extended condition.

FIG. 3 shows an exemplary implant sleeve in accordance with another exemplary embodiment of the invention in a non-extended condition.

FIG. 4 shows an exemplary implant sleeve illustratively in use with an exemplary muscle bundle.

FIG. 5 shows an exemplary implant sleeve illustratively in use with an exemplary muscle bundle.

FIG. 6 depicts a series of mandrels designed to produce the implant sleeve shown in FIG. 2.

FIG. 7 illustrates an exemplary process used to create polymer fibers that may be used to form an embodiment of an implant sleeve.

FIGS. 8A and 8B show scanning electron micrographs of a smooth polymer fiber (8A) and a textured polymer fiber (8B) that may be produced using the process illustrated in FIG. 7.

In the figures, unless otherwise noted, each of the scale marks represents 1 mm. While scales are illustrated in these figures, these are simply for reference and it is noted that the invention is not limited to the illustrated, exemplary sizing.

### DETAILED DESCRIPTION OF THE INVENTION

The detailed description set forth below in connection with the appended drawings is intended as a description of various and preferred embodiments of the present invention, and is not intended to represent the only forms that may be developed or utilized. The description sets forth the various functions in connection with the illustrated embodiments, but it is to be understood, however, that the same or equivalent functions may be accomplished by different embodiments that are also intended to be encompassed within the scope of the present invention.

The present invention provides a biocompatible sleeve for implantable muscles to repair injured or diseased muscles, particularly those that cannot be treated with autografts or allografts. The sleeve is produced in such a way that pressing the ends together along its longitudinal axis increases its diameter to facilitate the insertion of the engineered muscles into the sleeve, and pulling the ends of the sleeve during implantation decreases its diameter so that the sleeve fits securely over the engineered muscles.

Small muscle constructs have been engineered, which approximate the size of muscle fascicles and are long enough to replace native muscles, but are small in diameter. If the diameter of the muscle can be scaled up, these muscles could potentially be used to replace small muscles of the face or hand. In one embodiment, it is contemplated by the present invention to bundle small muscle constructs together in a sleeve in the form of a bioresorbable polymeric sleeve, formed by the textile processes of braiding, knitting, or weaving, with an expandable and contractible diameter. The sleeve diameter is expandable to allow multiple muscle constructs to be easily inserted into the sleeve, supporting scale up of the resulting muscle diameter. The sleeve diameter can be contracted to induce effective muscle to muscle contact. In addition, the sleeve provides sufficient mechanical integrity to the bundled muscle construct so that the encased muscle constructs can be anchored upon implantation. The sleeve is configured to transmit physiologic mechanical forces to the maturing tissue and to support neurovascular ingrowth from the patient.

The biocompatible sleeve in accordance with the present invention preferably is utilized with aligned engineered muscle tissue, intended for implantation in a patient, which is produced in vitro prior to bundling. Three-dimensional (3D) muscle constructs formed in vitro are preferably used in accordance with the present invention. These muscles differ from other reported engineered muscle in that 3D muscle constructs are fully formed and bundled together in a synthetic degradable sleeve. This sleeve provides the bundled muscle construct strength for implantation and mechanical support to the tissue until maturation is complete.

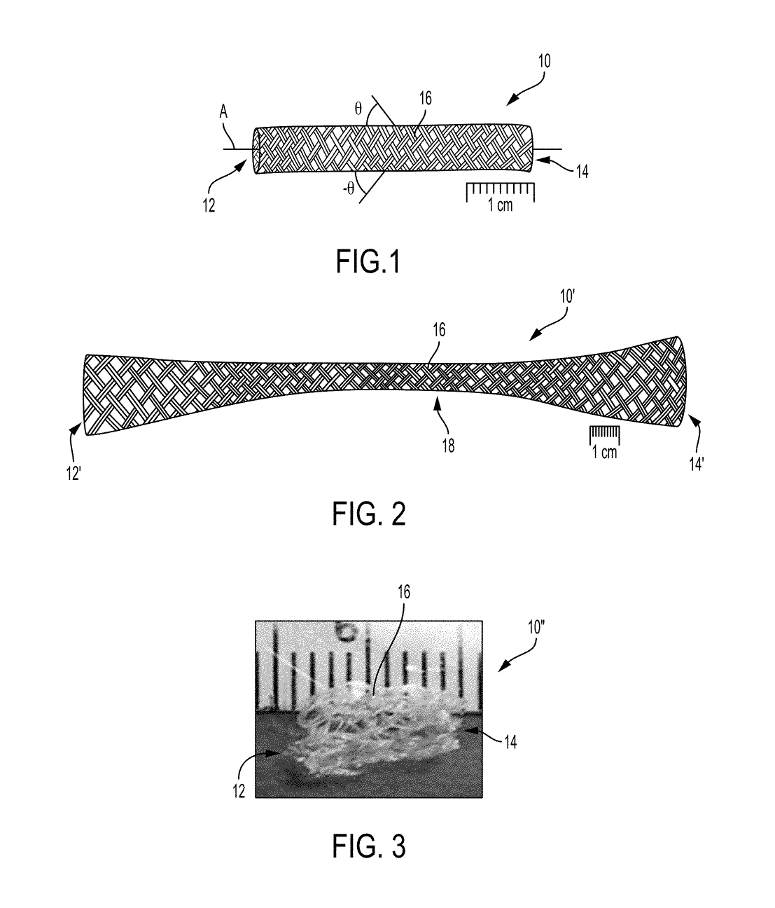

Referring to FIG. 1, an implant sleeve 10 in accordance with an exemplary embodiment of the invention will be described. The sleeve 10 has a cylindrical tube body with open ends 12, 14. The sleeve 10 of the present embodiment is formed by braiding polymeric strands 16. The strands 16 are preferably braided in a biaxial braid configuration such that the tube body has natural conformability, which refers to the diameter reduction when the tube is pulled lengthwise and diameter increase when the tube is pushed inward along the longitudinal axis A. By biaxial, some of the strands 16 extend at a first angle θ relative to the longitudinal axis A while the remaining strands 16 extend at a second, opposite angle −θ relative to the longitudinal axis A.

FIG. 2 illustrates an implant sleeve 10′ in accordance with another exemplary embodiment of the invention. The sleeve 10′ is substantially the same as the previous embodiment and includes biaxially braided strands 16, however, the ends 12′ 14′ are formed with an increased diameter compared to a central portion 18 of the tube body. In all other respects, the sleeve 10′ is the same as in the previous embodiment.

FIG. 3 illustrates an implant sleeve 10″ in accordance with yet another exemplary embodiment of the invention. The sleeve 10″ again has a tubular body with open ends 12, 14. The sleeve 10″ of the present embodiment is formed by knitting polymeric strands 16. The sleeve 10″ is knitted such that it also has natural conformability and functions in the same manner as the sleeve 10.

The sleeves are created for holding together the bundled muscle constructs. In a preferred embodiment, the material used for the sleeve is a copolymer with three components: desamniotyrosyl-tyrosine alkyl ester (DTE), desamniotyrosyl-tyrosine pendant free carboxylic acid (DT), and poly(ethylene glycol) (PEG). The concentration of each component can be adjusted to achieve the desired mechanical properties and the degradation rate for the sleeve.

The naturally conforming sleeve concept protects the immature engineered muscle constructs after insertion inside a permeable sleeve with mechanical properties matched to that of native muscle. The design of the sleeve allows for the encapsulation of small engineered muscle constructs.

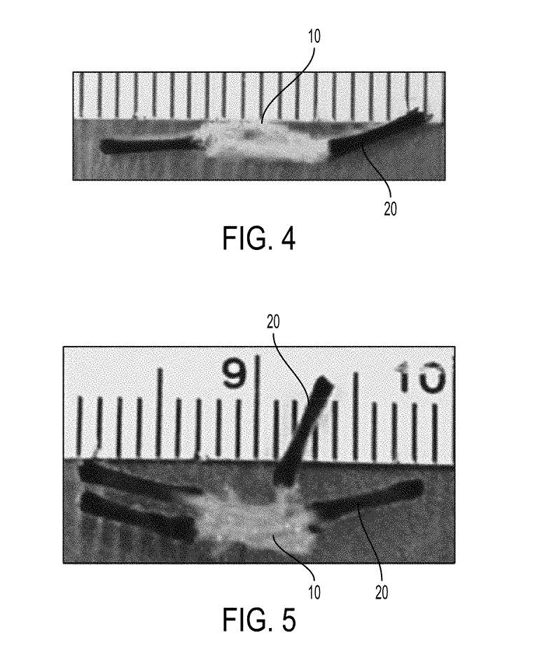

The polymeric sleeve designs may be configured to contain multiple muscle constructs. For example, FIG. 4 illustrates an exemplary sleeve 10 with a single muscle construct 20 positioned therein while FIG. 5 illustrates an exemplary sleeve 10 with two muscle constructs 20 positioned therein. The invention is not limited to the illustrated constructs, but instead, additional muscle constructs could be inserted as the sleeve diameter increases. Upon implantation, the sleeve will transmit physiological forces to the muscle construct, inducing fusion and maturation of the encased engineered muscle.

The preferred state of the bundled engineered muscle construct assembly is a collection of 5-20 engineered muscles cultured approximately 6-15 days in vitro after differentiation. These 3D engineered muscles have native-like tissue architecture and can have an endothelial network which forms along with the muscle. These networks connect with the patient blood vessels upon implantation.

Preferably, the cylindrical sleeve is formed from polymer fibers using a textile process, such as weaving, knitting or braiding. The fibers may be made from a polymer chosen from a family of tyrosine-derived polycarbonates so as to achieve the desired mechanical properties and the level of degradability in the implanted device. It is to be understood that any biocompatible degradable polymer can be used to form the fibers for purposes of the present invention. Examples of such polymers include, but are not limited to, poly(lactic acid), poly(glycolic acid), poly(lactic-co-glycolic acid), polycaprolactone, and polyanhydrides. The preferred composition of the fibers are poly(DTE-co-10% DT-co-01% PEG carbonate). Natural materials, for example, collagen, may alternatively or additionally be utilized.

In some embodiments, the biocompatible polymer composition is bioresorbable, biodegradable, or both. In some embodiments the polymer composition is radiopaque, whereas in other embodiments it is not radiopaque. In some embodiments, the polymer compositions comprise a substance such as, but not limited to, small molecules, bioactive compounds, or proteins, which may be dispersed in the polymer composition and/or covalently attached to the first polymer phase, the second polymer phase or both.

In an embodiment, the polymer has a modulus of elasticity in wet conditions between about 1 and about 20000 kPa, and preferably between about 100 and about 1500 kPa, as measured by standard tensile testing procedures that are well known to those of ordinary skill in the art. In some embodiments, the polymer fibers may have a shape memory.

The term “degradation” is defined as the process leading to the chemical cleavage of the polymer backbone, resulting in a reduction in polymer molecular weight and mechanical strength. The rate of polymer degradation under physiological conditions is predominantly determined by the type of bonds used to link the individual polymer repeat units together. Hence, polyanhydrides, e.g., polymers containing the highly labile anhydride linkage, will tend to degrade faster than polyesters. In contrast, the term “resorption” is defined as the process leading to a reduction of the mass of an implanted device. The rate of resorption is predominantly governed by the solubility of the polymer itself or its degradation products. The resorption of an implant is complete, once the entire mass of the implant has been removed from the implant site. Degradation and resorption do not always go hand-in-hand.

In an embodiment, polymers are selected so that degradation of the polymer structure is matched to the effective innervation and maturation of the bundled muscle for which the sleeve is being designed. The polymer structure should be tuned to degrade within a one to three months for small muscles without significant load. For larger muscles with significant loads, the degradation should occur over a period of several months up to a year. In addition, complex muscles may require a combination of sleeves to form the appropriate muscle architecture. Small bundles of muscle constructs will be encircled by polymer sleeves which degrade quickly within a few weeks to a month. These small bundles will be encircled by a larger polymer sleeve which provides overall support and degrades more slowly over a period of months to a year.

In an embodiment, polymers are selected having intrinsic physical properties appropriate for use in tissue sleeves with suitable rigidity, strength and degradation behavior. Such polymers include, if the polymer is amorphous, polymers with a glass transition temperature greater than 37° C. when fully hydrated under physiological conditions and, if the polymer is crystalline, a crystalline melting temperature greater than 37° C. when fully hydrated under physiological conditions.

In at least one embodiment, the polymer, the fiber diameter, filament number, and the braiding geometry is selected so that the stiffness of the sleeve matches that of the muscle. This is achieved in four steps: First a polymer is chosen so that the intrinsic stiffness of the polymer is adequate, between 0.5 to 4 Mpa, preferably about 2 MPa. Second, the polymer is spun into a fiber of a diameter so that the fibers are not too rigid. The preferred diameters are 40 to 100 μm. Third, the number of filaments to be bundled into a yarn is chosen so that the yarn is strong enough to fabricate a sleeve in the machine used for knitting/braiding/or weaving, but still flexible and light enough to yield a soft sleeve. The preferred number of filaments in a yarn is three. Fourth, the braid geometry, the angle of the braid and the number of spindles used during braiding is selected so that the braided sleeve has the stiffness that matches the stiffness of the muscle. Braiding is done either with 12 or 24 spindles, with 12 spindles preferred to obtain softer sleeves with more open structure. The braid angle is typically 30° to 45°.

In at least one embodiment, polymers are selected containing between approximately 5 and 30 mol % of monomers having solubility in phosphate buffered saline (PBS) under physiological conditions of greater than about 3 mg/mL to provide the desired rate of degradation and resorption. For purposes of the present invention, “physiological conditions” are defined as storage in PBS, at 0.1 M concentration, pH 7.4, and 37° C.

In at least one embodiment, polymers are selected which degrade and/or resorb within a predetermined time. For this reason, embodiments according to the present invention include polymers with molar fractions of monomeric repeating units with pendant fee carboxylic acid groups, such as DT, between about 0 and about 30 mol %, and preferably between about 5 and about 20 mol %.

Poly(alkylene glycol) segments, such as PEG, decrease the surface adhesion of the polymers. By varying the molar fraction of poly(alkylene glycol) segments in the block copolymers provided by the present invention, the hydrophilic/hydrophobic ratios of the polymers can be changed to adjust the ability of the polymer coatings to modify cellular behavior. Increasing levels of poly(alkylene glycol) inhibit cellular attachment, migration and proliferation. Secondarily, PEG increases the water uptake, and thus increases the rate of degradation of the polymer. Accordingly, in an embodiment, polymers are selected in which the amount of poly(alkylene glycol) is limited to between 0 and about 15 mol %, and preferably between about 0.5 and about 5 mol %. The poly(alkylene glycol) may have a molecular weight of 1 k to 2 k.

Polymers according to the present invention include polyethers, polyurethanes, polycarbamates, polythiocarbonates, polycarbonodithionates and polythio-carbamates. Polycarbonates, specifically poly(amide carbonates), as well as polyurethanes, polycarbamates, polythiocarbonates, polycarbonodithionates and polythiocarbamates are prepared by the process disclosed by U.S. Pat. No. 5,198,507, the disclosure of which is incorporated by reference. Polyesters, specifically poly(ester amides), are prepared by the process disclosed by U.S. Pat. No. 5,216,115, the disclosure of which is incorporated herein by reference. Polyiminocarbonates are prepared by the process disclosed by U.S. Pat. No. 4,980,449, the disclosure of which is incorporated by reference. Polyethers are prepared by the process disclosed by U.S. Pat. No. 6,602,497, the disclosure of which is incorporated by reference.

The polycarbonate polymers of the present invention are disclosed in U.S. Pat. Nos. 6,120,491 and 6,475,477, the disclosures of which are incorporated herein by reference. The polycarbonates may also be prepared by dissolving the monomers in methylene chloride containing 0.1M pyridine or triethylamine. A solution of phosgene in toluene at a concentration between 10 and 25 wt %, and preferably about 20 wt %, is added at a constant rate, typically over about two hours, using a syringe pump or other means. The reaction mixture is quenched by stirring with tetrahydrofuran (THF) and water, after which the polymer is isolated by precipitation with isopropanol. Residual pyridine (if used) is then removed by agitation of a THF polymer solution with a strongly acidic resin, such as AMBERLYST 15.

The polyarylate polymers of the present invention are also disclosed in U.S. Pat. No. 6,120,491 and are prepared by the direct reaction of diphenols with aliphatic or aromatic dicarboxylic acids in the carbodiimide mediated process disclosed by U.S. Pat. No. 5,216,115 using 4-(dimethylamino) pyridinium-p-toluene sulfonate (DPTS) as a catalyst. The disclosure of U.S. Pat. No. 5,216,115 is incorporated herein by reference.

Polymers with at least one bromine- or iodine-substituted aromatic ring are radio-opaque, such as the polymers prepared from radiopaque diphenol compounds prepared according to the disclosure of U.S. Pat. No. 6,475,477, the disclosure of which is incorporated herein by reference. The polymer glass transition temperature tends to increase as the degree of halogenation.

It is understood that the foregoing properties may be provided by a single polymer or by combinations of two or more polymers in the presently described sleeve.

In accordance with the present invention, the polymer fibers used to form the sleeves may be smooth or textured. In one embodiment, suitable texture can be imprinted on extruded fibers by using the technique of demixing. The fiber is coated with a thin film of a blend of two immiscible polymers; the coating is typically 100 nm to 10 μm thick, and preferably 0.5 to 5 μm, thick. The two polymers are dissolved in a common solvent to facilitate coating. Examples of blends that have been investigated are: polystyrene and poly(DTE carbonate) in tetrahydrofuran, polystyrene and poly(methyl methacrylate) in tetrahydrofuran, and poly(ethylene glycol) and polycaprolactone in a mixture of tetrahydrofuran and N,N-dimethylformamide. One of the polymers is sacrificed by exposing the fiber to a suitable solvent (e.g., cyclohexane for polystyrene and water for poly(ethylene glycol)), which is not a solvent for the second polymer. The fiber will develop a texture depending on the concentration and composition of the polymer solution, and the manner in which it was coated. Such surface textures are believed to impact cell response.

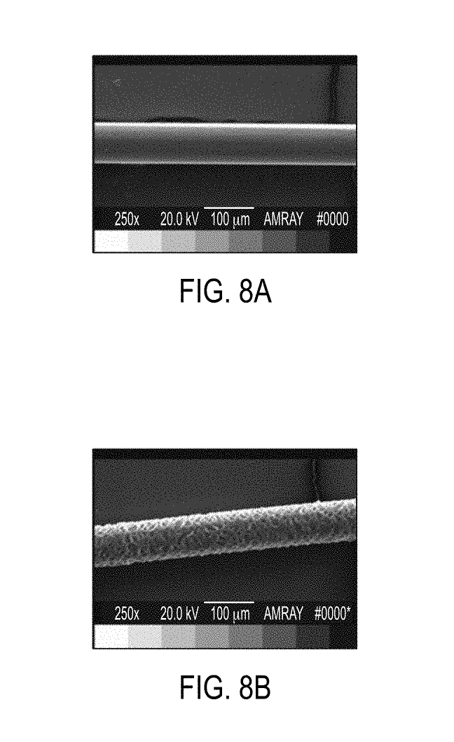

A continuous process shown in FIG. 7 can be used to produce such textured fibers. An example of a smooth polymer fiber not subjected to such process is shown in FIG. 8A. An example of the textured product obtained by such process is shown in FIG. 8B. Polymer fibers with surface textures are believed to have advantages over smooth filaments. It is anticipated they will retain particulate matter in the crevices of the pattern. It is also believed they influence cell attachment, growth, proliferation, and differentiation. In addition to its use with the sleeves of the present invention, this texturing process may be used produce fibers useful in other structures fabricated using textile processes having utility in a variety of implantable medical devices.

In another embodiment, polymer fibers can also be textured by passing the fiber through a bath of a suitable solvent such as tetrahydrofuran, dimethyl formamide, and dichloromethane for a suitable time (1 sec to 10 sec), and immediately drying the fiber. The solvent will etch the fiber, and drying will leave this textured pattern on the fiber. Such surface textures are known to control cell response. This can be achieved using the scheme in FIG. 7 where, instead of two-pass process, the process consists of a single pass through the solvent bath.

The sleeve may also include elements that help maintain the sleeve's structural integrity. For example, the sleeve may be constructed as to be prevented from pinching when it is bent during implantation. In certain embodiments, this may be accomplished by selectively filling the hollow interior of the sleeve with polymer fibers, such as those used in constructing the sleeve itself. Alternatively, the interior of the sleeve may be selectively filled with a gel composed of, for example, collagen or polymer. The sleeve may be modified with such polymer fibers or gels either prior to or after insertion of the muscle construct bundle.

FIG. 2 illustrates an embodiment of the sleeve that has been constructed so as to include flared ends. The flared ends will assist with muscle construct insertion and will allow the sleeve to slip over the muscle suture anchors when stretched. This illustrated embodiment is braided from 24 spools of yarns made from three filaments each having a 60 μm diameter. The polymer fibers are preferably manufactured by melt spinning, but may be formed using any known method in the art. The ends of the sleeves may be sealed with a quick setting biocompatible cyanoacrylate adhesive, or by using a hot knife (Temp ˜100° C., duration about 2 seconds), to prevent the sleeve from unraveling.

In a preferred embodiment, the polymer fibers that form the sleeve may be woven so as to form an expandable/collapsible design. In this embodiment, the diameter of the sleeve increases when the ends of the sleeves are pushed towards each other to facilitate the insertion of the bundled tissue into the sleeve. Once inserted, the ends of the sleeves may be pulled away from each other to decrease the diameter of the sleeve, thus creating a secure fit around the bundled tissue.

An inert, soluble material like agarose or gelatin can be used to temporarily glue the sleeve open in the compressed position, to assist with inserting the muscle constructs into the sleeve. After the muscle constructs are inserted, the temporary glue can be removed by dissolved by immersing the muscle construct-sleeve bundle in saline.

As an alternative to inserting the assembled muscle bundles into the sleeve through the ends, the sleeves can be slit open along the sleeve length using a thermal cutter. After placing the muscle construct bundle into the open sleeve, the longitudinal cut edges can be sealed using a biocompatible adhesive such a fibrin.

The sleeve of the present invention is preferably biodegradable, and has a specific degradation profile that may be tuned depending on the desired application. In certain embodiments, the degradation profile may be controlled by forming the sleeve from multiple polymer fibers, each having a different degradation profile. The degradation profile of the sleeve may be further tuned by coating the formed sleeve with an electrospun polymer fiber mat.

The sleeve may also be configured to elute bioactive molecules, including drugs that would be useful post-implantation. In certain embodiments, this may be accomplished by forming the sleeve from drug-eluting polymer fibers. In other embodiments, a drug-polymer coating may be applied to a formed sleeve.

While the present invention has been described with respect to its use in the implantation of muscle tissue, it should be understood that the presently described sleeve may also be utilized to facilitate implantation of other tissue types in both humans and animals.

The following examples are presented to illustrate the nature of the invention. The present invention, however, should not be considered as being limited to the details therein.

### EXAMPLES

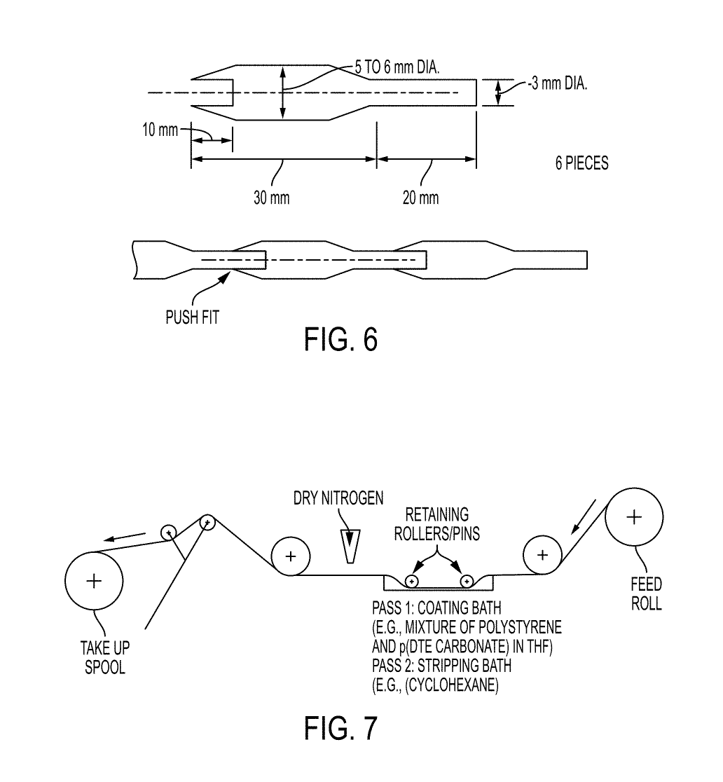

The design was implemented with a rapidly (12 weeks) degradable poly(DTE-co-10% DT-co-2% PEG carbonate). Fibers were melt-spun, drawn to the desired diameter (50-100 μm), and bundled into yarns with 3-7 filaments per yarn. The sleeves are made using a braiding machine consisting of 24 spools. Straight sleeves were obtained using a cylindrical mandrel, and flared sleeves were obtained using a specially machined mandrel so that multiple sleeves can be braided in a single run (FIG. 6). The ends of each piece of sleeve were thermally sealed using a hot knife. To minimize inflammation, the sleeves were cleaned in a sonicator with cyclohexane, followed by 5% Tween 20, a detergent, and then washed three times in DI water. These sleeves were sterilized with UV radiation, and subcutaneously implanted in Sprague-Dawley rats to assess tissue response. The implants were harvested after three weeks, and stained with hematoxylin and eosin (H & E) to assess inflammation. In a separate study, single fibers and empty sleeves were implanted subcutaneously into Wistar rats to assess the biocompatibility of the material. Minimal inflammatory response was observed at 1, 3, and 5 weeks when the polymer fibers were properly cleaned and sterilized. Little inflammatory infiltrate and few giant cells were observed within the sleeves.

Additional sleeves were made using the above procedure and implanted into C57BL/6 mice. If the sleeves were too stiff, the fibers protruded through the surrounding tissue, causing irritation to the mouse. Therefore, smaller diameter filaments (30-60 μm) were extruded to decrease overall stiffness. After forming three-filament yarns, sleeves were fabricated by braiding or knitting. Knitted sleeves were softer than braided sleeves. But it was far more difficult to insert a bundle of muscle constructs into a knitted sleeve than into a braided sleeve.

Sleeves containing engineered muscle constructs as well as their controls were implanted in the fat pad on the back of five immunodeficient nude mice as follows:

Braided sleeve enclosing 1 engineered muscle construct

Braided sleeve enclosing 2 engineered muscle constructs

1 engineered skeletal construct

1 empty braided sleeve

1 empty knitted sleeve

Knitted and braided sleeves were cut to a length which allowed multiple muscle constructs to be inserted. Muscle constructs were inserted only into braided sleeves as insertion into knitted sleeves was difficult. For biocompatibility assessment, the implants listed above were enclosed in the fat pads of nude mice, one implant per mouse; the fat pads were closed with a 6-0 suture to keep the implants in place. The skin was closed with 6-0 sutures. The nude mouse implanted with the empty knitted sleeve died 2 d after implantation due to anesthesia complications. The other implants along with their fat pads and underlying tissue were explanted at 2 wk post-implantation. These explants were embedded in paraffin, sectioned, and stained for Hematoxylin and Eosin (H & E) as well as a macrophage marker (CD68).

In the empty braided sleeve, moderate inflammatory infiltrate was observed surrounding the fibers and within the sleeve. The fibers of the sleeve were surrounded by vascularized loose connective tissue, containing CD68+ macrophages, and fat. As expected, giant cells were located close to the fibers (left, bottom panel: yellow arrow).

In the engineered skeletal muscle construct implanted alone in the fat pad, mild inflammation was observed, with a few inflammatory cells associated with the closing 6-0 sutures. No giant cells were present.

In the braided sleeve with one engineered skeletal muscle construct, the sleeve was filled with fat tissue and vascularized loose connective tissue (lower left panel, blue arrow); there was moderate inflammation with inflammatory cells. A few giant cells associated with the fibers (lower left panel, white arrow) were present. The engineered muscle construct contained some small blood vessels with red blood cells (lower left panel, red arrow).

In the braided sleeve with two engineered skeletal muscle constructs, the sleeve was filled with fat tissue and vascularized loose connective tissue (lower left panel, blue arrows); there was moderate inflammation in the explant with CD68+ macrophages. Giant cells associated only with the fibers (lower left panel, white arrows) were present. The engineered muscle constructs contained several small blood vessels with red blood cells (lower left panel, red arrow).

In summary, the engineered muscle constructs were easily inserted into braided sleeves. The sleeves induced a moderate inflammatory response, as denoted by the presence of CD68+ macrophages. Several macrophages were observed within muscle constructs enclosed within braided sleeves; the viability of the engineered muscle constructs was not impacted by the sleeve.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A biocompatible sleeve for use with implantable tissue, the sleeve comprising a cylindrical, helically wound, sleeve fabricated by braiding, knitting or weaving, and comprising polymer fibers, wherein said polymer fibers comprise a first resorbable biocompatible polymer.

2. The biocompatible sleeve of claim 1, wherein the diameter of said sleeve increases when the ends are pushed towards each other along the longitudinal axis, and the diameter decreases when the ends are pulled away from each other along the longitudinal axis.

3. The biocompatible sleeve of claim 2, wherein the polymer fibers are braided in a biaxial braid configuration.

4. The biocompatible sleeve of claim 1, wherein said sleeve is in the form of a conduit or sheath.

5. The biocompatible sleeve of claim 1, wherein said polymer fibers have a shape memory.

6. The biocompatible sleeve of claim 1, wherein said first resorbable biocompatible polymer comprises:
(a) desamniotyrosyl-tyrosine alkyl ester (DTE),
(b) desamniotyrosyl-tyrosine free carboxylic acid (DT), and
(c) poly(alkylene glycol).

7. The biocompatible sleeve of claim 6, wherein said poly(alkylene glycol) comprises poly(ethylene glycol) (PEG).

8. The biocompatible sleeve of claim 1, wherein said implantable tissue is engineered muscle tissue.

9. The biocompatible sleeve of claim 1, wherein said polymer fibers comprise textured polymer fibers.

10. The biocompatible sleeve of claim 1, wherein said sleeve further comprises a collagen gel.

11. The biocompatible sleeve of claim 1, wherein said sleeve further comprises a polymer gel.

12. The biocompatible sleeve of claim 1, wherein said sleeve further comprises a coating formed from an electrospun mat.

13. The biocompatible sleeve of claim 1, wherein said sleeve further comprises a second polymer having a degradation profile different from that of said first polymer.

14. The biocompatible sleeve of claim 1, wherein said sleeve further comprises a drug.

15. An implant comprising tissue encased in a sleeve of claim 1.

16. The implant of claim 15, wherein said tissue is muscle tissue.

17. A method of repairing mammalian muscles in a subject in need thereof, comprising the steps of:
encasing muscle tissue in the biocompatible sleeve of claim 1 to form an encased muscle implant; and
implanting said encased muscle implant in said subject.

18. The method of claim 17, wherein said muscle tissue is fully formed 3D muscle.

19. The method of claim 17, wherein said muscle construct is formed in vitro.
